(19) United States
(12) Patent Application Publication
(10) Pub. No.: US 2021/0249714 A1
(43) Pub. Date: Aug. 12, 2021

(54) Lithium Air Secondary Battery

(71) Applicant: Nippon Telegraph and Telephone Corporation, Tokyo (JP)

(72) Inventors: Masahiko Hayashi, Musashino-shi, Tokyo (JP); Masaya Nohara, Musashino-shi, Tokyo (JP); Shuhei Sakamoto, Musashino-shi, Tokyo (JP); Mikayo Iwata, Musashino-shi, Tokyo (JP); Takeshi Komatsu, Musashino-shi, Tokyo (JP)

(21) Appl. No.: 16/980,311

(22) PCT Filed: Mar. 14, 2019
(86) PCT No.: PCT/JP2019/010468

§ 371 Date: Sep. 11, 2020

(30) Foreign Application Priority Data
Mar. 16, 2018 (JP) 2018-048848

(51) Int. Cl. H01M 12/02; H01M 12/08; C07D 273/01; C07D 273/02

(52) U.S. Cl. CPC H01M 12/02; H01M 12/08; H01M 2300/0025; C07D 273/02; C07D 273/01

(57) ABSTRACT

A lithium air secondary battery is allowed to operate as a high-capacity secondary battery, thereby implementing high output and a large discharge capacity. A lithium air secondary battery 100 includes an air electrode 102 using oxygen in the air as a positive electrode active material, a negative electrode 104 using metallic lithium or a lithium-containing material as a negative electrode active material, and an organic electrolyte 106 placed between the air electrode 102 and the negative electrode 104, and including a lithium salt. The organic electrolyte 106 includes a crown ether compound having an azo group as an additive.

### TECHNICAL FIELD

The present invention relates to a technology of a lithium air secondary battery. More particularly, the present invention relates to a lithium air secondary battery which is smaller in size and more lightweight than conventional secondary batteries such as a lead storage battery and a lithium ion battery, and can implement far larger discharge capacity.

### BACKGROUND ART

Reportedly, a lithium air secondary battery using oxygen in the air as a positive electrode active material is constantly supplied with oxygen from the outside of the battery, and can be filled with a large amount of metallic lithium of a negative electrode active material in the battery, and hence exhibits a very large discharge capacity per unit volume of the battery.

In NPL 1 and 2, the battery performances such as the discharge capacity, and charging and discharging cycling characteristics are tried to be improved by adding various catalysts to the air electrode of the positive electrode. For example, a transition metal oxide has been studied as the electrode catalyst of the air electrode. In NPL 1, the transition metal oxide such as λ-MnO₂ is studied. In NPL 2, transition metal oxides such as iron oxide (Fe₂O₃) and cobalt oxide (Co₃O₄) are mainly studied.

### CITATION LIST

### Non Patent Literature

[NPL 1] J, Read, “Characterization of the Lithium/Oxygen Organic Electrolyte Battery”, Journal of The Electrochemical Society, 149(9), Vol. 149, 2002, p. A1190-p. A1195

[NPL 2] Aurelie Debart, and three other members, “An O2 cathode for rechargeable lithium batteries: The effect of a catalyst”, Journal of Power Sources, Vol. 174, 2007, p. 1177-p. 1182

### SUMMARY OF THE INVENTION

### Technical Problem

However, with the lithium air secondary battery disclosed in NPL 1, although the charging and discharging cycle is possible, the discharge capacity is reduced to about ¼ after four cycles. This unfavorably results in the low performances as a secondary battery. Further, with the lithium air secondary battery of NPL 1, the charging voltage is about 4.0 V, and is much larger than 2.7 V of the average discharging voltage. This also unfavorably results in a low charging and discharging energy efficiency.

On the other hand, in NPL 2 nine kinds of catalysts are studied, and the discharging capacity as very large as 1000 to 3000 mAh/g per weight of carbon included in the air electrode can be obtained. However, when charging and discharging are repeatedly performed, the reduction of the discharge capacity is remarkable. For example, in the case of Co₃O₄, the capacity retention rate becomes about 65% in 10 cycles. In this manner, also with the lithium air secondary battery of NPL 2, remarkable reduction of the capacity is observed, and sufficient characteristics as a secondary battery cannot be obtained. Further, in a large number of measurement results, while the average discharging voltage is about 2.5 V, the charging voltage shows 4.0 to 4.5 V, and even the lowest is about 3.9 V. For this reason, also for the lithium air secondary battery of NPL 2, the charging and discharging energy efficiency is low.

Incidentally, according to much literature including NPL 1 and 2, as the organic electrolyte of a lithium air secondary battery, a solution obtained by dissolving a lithium salt such as LiClO₄, LiPF₆, or LiTFSI (lithium bistrifluoromethanesulfonyl imide) in a carbonic acid ester type solvent such as propylene carbonate, a glyme type solvent such as tetraethylene glycol dimethyl ether (TEGDME), or a sulfoxide type solvent such as dimethyl sulfoxide (DMSO) in a concentration of about 1.0 mol/l is used.

The present invention was completed in view of the foregoing circumstances. It is an object of the present invention to achieve a high output and a large discharge capacity by causing a lithium air secondary battery to operate as a high-capacity secondary battery.

### Means for Solving the Problem

In order to solve the problems up to this point, a lithium air secondary battery in accordance with claim 1 includes a positive electrode using oxygen as a positive electrode active material; a negative electrode using metallic lithium or a lithium-containing material as a negative electrode active material; and an organic electrolyte placed between the positive electrode and the negative electrode, and including a lithium salt, wherein the organic electrolyte includes a crown ether compound having an azo group.

The lithium air secondary battery according to claim 2 includes the crown ether compound that is any of 1-aza-15-crown-5-ether, 1-aza-18-crown-6-ether, 4,13-diaza-18-crown-6-ether, N,N′-dibenzyl-4,13-diaza-18-crown-6-ether, and N-phenyl aza-15-crown-5-ether in the lithium air secondary battery according to claim 1.

### Effects of the Invention

In accordance with the present invention, it is possible to provide a lithium air secondary battery having a large discharge capacity and excellent charging and discharging cycling performances.

### BRIEF DESCRIPTION OF DRAWINGS

FIG. 1 is a view showing a configuration of a lithium air secondary battery.

FIG. 2 is a view showing a cross sectional structure of a cylindrical lithium air secondary battery.

FIG. 3 is a view showing a structural formula of compound 1.

FIG. 4 is a view showing a structural formula of compound 2.

FIG. 5 is a view showing a structural formula of compound 3.

FIG. 6 is a view showing a structural formula of compound 4.

FIG. 7 is a view showing a structural formula of compound 5.

FIG. 8 is a view showing initial discharging and charging curves of Example 1.

FIG. 9 is a view showing the battery performance test results of Examples 1 to 5.

FIG. 10 is a view showing a structural formula of compound 6.

FIG. 11 is a view showing the battery performance test results of Comparative Examples 1 and 2.

### DESCRIPTION OF EMBODIMENTS

Below, referring to the accompanying drawings, a lithium air secondary battery in accordance with one embodiment of the present invention will be described in details. The present invention is not construed only based on the following embodiments, and can be executed by being appropriately changed within the scope not changing the gist thereof.

### Configuration of Lithium Air Secondary Battery

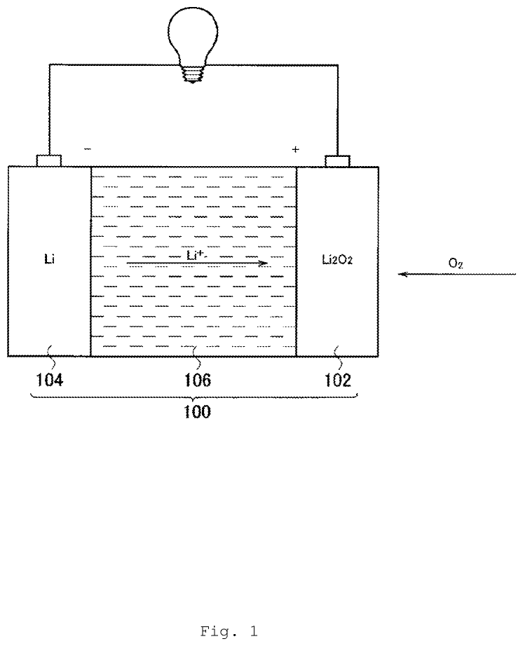

FIG. 1 is a view showing a configuration of a lithium air secondary battery in accordance with the present embodiment. A lithium air secondary battery 100 includes at least an air electrode 102, a negative electrode 104, and an organic electrolyte 106. The air electrode 102 functions as a positive electrode. Further, the organic electrolyte 106 is placed between the air electrode 102 and the negative electrode 104. The organic electrolyte 106 includes an azo type crown ether compound having an azo group as an additive.

The air electrode 102 can include a catalyst and a conductive material as constitutional element. Further, the air electrode 102 preferably includes a binding agent for integrating the conductive material. Further, the negative electrode 104 can include metallic lithium or a substance material such as a lithium-containing alloy capable of releasing or absorbing lithium ions as a constitutional element.

Below, the constitutional elements forming a lithium air secondary battery will be described.

(I) Organic Electrolyte 106

The organic electrolyte 106 includes at least an azo type crown ether compound as an additive. More specifically, the organic electrolyte 106 includes a lithium salt and an organic solvent, and includes an azo type crown ether compound as an additive. The amount of the additive to be added based on the amount of the organic electrolyte 106 desirably falls within the range of 0.001 to 1 wt %.

It is essential only that the organic electrolyte 106 is a substance in which lithium ions can move between the air electrode 102 and the negative electrode 104, and it is essential only that a nonaqueous solvent including a metal salt containing lithium ions dissolved therein can be used.

As such a solute, for example, lithium hexafluorophosphate (LiPF₆), lithium perchlorate (LiClO₄), and lithium bistrifluoromethane sulfonyl imide [(CF₃SO₂)2NLi] (LiTFSA) can be used.

Further, as the solvent, for example, carbonic acid ester type solvents such as dimethyl carbonate (DMC), methyl ethyl carbonate (MEC), methyl propyl carbonate (MPC), methyl isopropyl carbonate (MIPC), methyl butyl carbonate (MBC), diethyl carbonate (DEC), ethyl propyl carbonate (EPC), ethyl isopropyl carbonate (EIPC), ethyl butyl carbonate (EBC), dipropyl carbonate (DPC), diisoprpyl carbonate (DIPC), dibutyl carbonate (DBC), ethylene carbonate (EC), propylene carbonate (PC), and 1,2-butylene carbonate (1,2-BC), an ether type solvent such as 1,2-dimethoxyethane (DME), a lactone type solvent such as γ-butyrolactone (GBL), a glyme type solvent such as tetraethylene glycol dimethyl ether (TEGDME), or a sulfoxide type solvent such as dimethyl sulfoxide (DMSO), or a solvent obtained by mixing two or more thereof can be used. The mixing ratio for using a mixed solvent has no particular restriction.

(II) Air Electrode (Positive Electrode) 102

The air electrode 102 includes at least a conductive material, and if required, can include a catalyst, a binding agent, and the like. Further, the air electrode 102 uses oxygen in the air as a positive electrode active material.

(II-1) Conductive Material

The conductive material included in the air electrode 102 is preferably carbon. Particularly, as the conductive materials, carbon blacks such as ketjen black and acetylene black, active carbons, graphites, carbon fibers, carbon sheet, carbon cloth, and the like can be used. However, these are not exclusive. Further, as the carbons, for example, commercially available products can be used, or existing products may be synthesized and formed.

(II-2) Catalyst

The catalyst of the air electrode 102 may be desirably an oxide catalyst highly active to both the reactions of oxygen reduction (discharge) and oxygen generation (charge) of manganese oxide (MnO₂), ruthenium oxide (RuO₂), or the like, and has no particular restriction so long as it is a conventionally known oxide catalyst. Specifically, single oxides such as MnO₂, Mn₃O₄, MnO, FeO₂, Fe₃O₄, FeO, CoO, Co₃O₄, NiO, NiO₂, V₂O₅, and WO₃, and composite oxides having a perovskite type structure such as La_{0.6}Sr_{0.4}MnO₃, La_{0.6}Sr_{0.4}FeO₃, La_{0.6}Sr_{0.4}CoO, La_{0.6}Sr_{0.4}CoO₃, Pr_{0.6}Ca_{0.4}MnO₃, LaNiO₃, and La_{0.6}Sr_{0.4}Mn_{0.4}Fe_{0.6}O₃ can be used. The catalysts can be synthesized using a known process such as a solid phase method or a liquid phase method.

Further, as the catalyst to be added to the air electrode 102, there can also be used a macrocyclic metal complex such as porphyrin or phthalocyanine including at least one of transition metals such as Mn, Fe, Co, Ni, V, and W at the central metal. The metal complexes may be mixed with carbon, followed by being subjected to a heat treatment in an inert gas atmosphere for activation.

As the catalysts to be added to the air electrode 102, not only the compound type, but also noble metals such as Pt, Au, and Pd, and simple substance metals of transition metals such as Co, Ni, and Mn may be used. For example, by allowing the metal to be carried in a highly dispersed manner on carbon, a high activity can be exhibited.

In the air electrode 102, at the three-phase part of the electrolyte (organic electrolyte 106)/electrode catalyst/gas (oxygen), the electrode reaction proceeds. Namely, the organic electrolyte 106 is permeated into the air electrode 102, and simultaneously, the oxygen gas in the atmosphere is supplied. As a result, the three-phase part including electrolyte-electrode catalyst-gas (oxygen) coexisting therein is formed. When the electrode catalyst is highly active, oxygen reduction (discharging) and oxygen generation (charging) smoothly proceed, resulting in a large improvement of the battery performances.

The discharging reaction at the air electrode 102 can be expressed as formula (1).

2Li⁺+O₂+2e⁻→Li₂O₂ (1)

The lithium ions (Li⁺) of formula (1) are ions which have dissolved in the organic electrolyte 106 from the negative electrode 104 by electrochemical oxidation, and have moved in the organic electrolyte 106 to the surface of the air electrode 102. Further, oxygen (O₂) has been incorporated into the inside of the air electrode 102 from the atmosphere (air). Incidentally, the material (Li⁺) dissolved from the negative electrode 104, the material (Li₂O₂) precipitated at the air electrode 102, and the oxygen (O₂) incorporated into the air electrode 102 are shown with the constitutional elements of FIG. 1.

With the lithium air secondary battery 100 of the present embodiment, in order to increase the battery reaction rate, a larger number of reaction sites (the three-phase parts of the electrolyte/electrode catalyst/air (oxygen)) for effecting the electrode reaction are desirably present. From such a viewpoint, in the present embodiment, it is important that the three-phase parts are present in a large amount at the surface of the electrode catalyst. The catalyst to be used preferably has a higher specific surface area. For example, the specific surface area after sintering is preferably 10 m²/g or more.

(II-3) Binding Agent (Binder)

The air electrode 102 can include a binding agent (binder). The binding agent has no particular restriction. Examples thereof may include polytetrafluoroethylene (PTFE), polyvinylidene fluoride (PVDF), and polybutadiene rubber. The binding agents can be each used as a powder or as a dispersion.

(II-4) Preparation of Air Electrode 102

The air electrode 102 can be prepared, for example, in the following manner. An oxide powder of a catalyst, a carbon powder, and a binder powder such as polyvinylidene fluoride (PVDF) are mixed in prescribed amounts. The resulting mixture is crimped on a support such as a titanium mesh. As a result, the air electrode 102 can be formed.

Alternatively, the mixture is dispersed in a solvent such as an organic solvent, resulting in a slurry. The resulting slurry is coated on a metal mesh, a carbon cloth or a carbon sheet for drying. As a result, the air electrode 102 can be formed.

Further, in order to increase the strength of the air electrode 102, and to prevent the leakage of the electrolyte, not only cold press but also hot press is applied. This can also manufacture the air electrode 102 more excellent in stability.

Incidentally, for the air electrode 102, one surface of the electrode forming the air electrode 102 itself is exposed to the atmosphere, and another surface is in contact with the electrolyte.

(III) Negative Electrode 104

The negative electrode 104 includes a negative electrode active material. The negative electrode active material has no particular restriction so long as it is a material which can be used as the negative electrode material of the lithium air secondary battery 100. For example, metallic lithium can be used. Alternatively, as a lithium-containing substance, a substance capable of releasing and occluding lithium ions may be used. Other than these, examples thereof may include an alloy of lithium and silicon or tin, or lithium nitride such as Li_{2.6}Co_{0.4}N.

The negative electrode 104 can be formed by a known method. For example, when a lithium metal is used for the negative electrode, the negative electrode 104 may be desirably manufactured by stacking a plurality of sheets of metallic lithium foils, and forming the foil into a prescribed shape.

The discharging reaction at the negative electrode 104 can be expressed as formula (2).

(Discharging Reaction)

Li→Li⁺+e⁻ (2)

Incidentally, during charging, the precipitation reaction of lithium of the opposite reaction of formula (2) is effected.

(IV) Other Constitutional Elements

The lithium air secondary battery 100 can include, in addition to the constitutional elements, structural members such as a separator, a battery case, and a metal mesh (e.g., titanium mesh), and other elements required of the lithium air secondary battery 100. For these, conventionally known ones can be used.

(V) Preparation of Lithium Air Secondary Battery

The lithium air secondary battery 100 is configured so as to include, as described above, at least the air electrode 102, the negative electrode 104, and the organic electrolyte 106, and such that, as shown in FIG. 1, the organic electrolyte 106 containing an azo type crown ether compound is interposed between the air electrode 102 and the negative electrode 104. The lithium air secondary battery 100 having such a configuration can be prepared in the same manner as with a conventional type secondary battery.

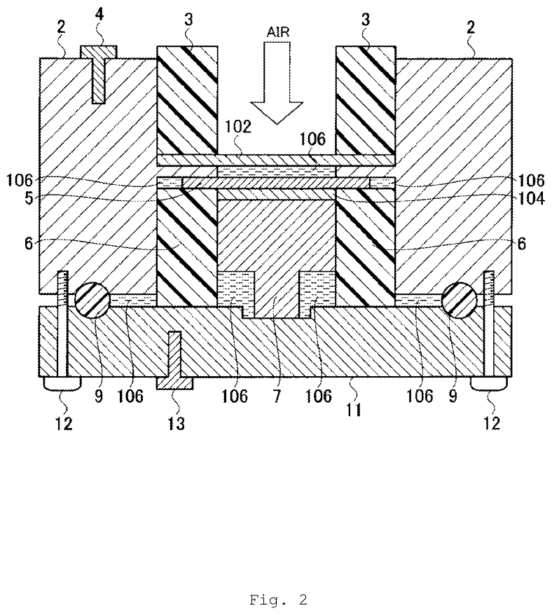

For example, the cylindrical lithium air secondary battery 100 as shown in FIG. 2 can be prepared. Specifically, first, the air electrode 102 is placed and fixed in the inside of the insulating coated (PTEF coated) air electrode support 2. The negative electrode 104 is fixed with respect to the negative electrode support 11. The internal space of the air secondary battery (the space between the air electrode 102 and the negative electrode 104) is filled with the organic electrolyte 106 including an azo type crown ether compound. Thus, the negative electrode support 11 is put thereon so that the negative electrode 104 is placed at the surface opposite to the surface in contact with the atmosphere of the air electrode 102, thereby fixing the whole air secondary battery.

In addition to the constitutional elements, members such as the separator 5 can be placed at the part lying between the air electrode 102 and the negative electrode 104. Additionally, an insulating member, an O ring 9, fixtures (an air electrode fixing PTFE ring 3, a negative electrode fixing PTFE ring 6, a negative electrode fixing washer 7, an insulating coated (PTEF coated) cell fixing screw 12), an air electrode terminal 4, a negative electrode terminal 13, and the like can be appropriately placed.

### EXAMPLES

### Preparation of Organic Electrolytes (TEGDME Solvents) Including Compounds 1 to 5

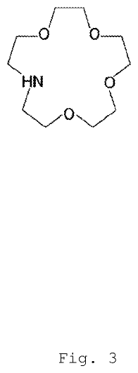

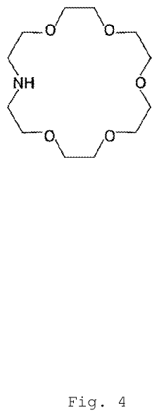

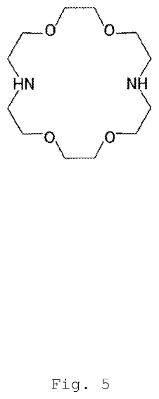

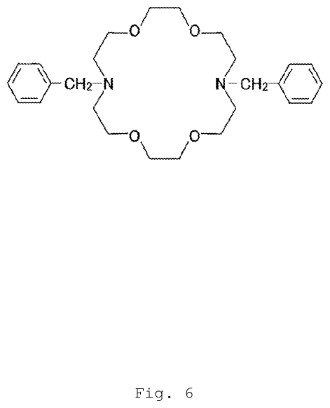

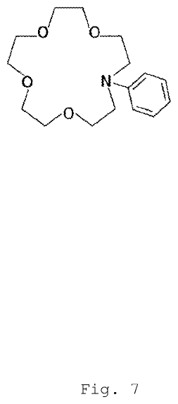

In the present example, the organic electrolyte 106 includes an azo type crown ether compound as an additive. Specifically, any compound of the following compounds 1 to 5 is included in the organic electrolyte 106. Respective structural formulas of the compounds 1 to 5 are shown in FIG. 3 to FIG. 7, respectively. Respective examples corresponding to the compounds 1 to 5 are referred to as Examples 1 to 5, respectively.

(Compound 1) 1-aza-15-crown-5-ether, (CAS No: 66943-05-3), Mw 219.28

(Compound 2) 1-aza-18-crown-6-ether, (CAS No: 33941-15-0), Mw 263.33

(Compound 3) 4,13-diaza-18-crown-6-ether, (CAS No: 23978-55-4), Mw 262.35

(Compound 4) N,N′-dibenzyl-4,13-diaza-18-crown-6-ether, (CAS No: 69703-25-9), Mw 442.60

(Compound 5) N-phenyl aza-15-crown-5-ether, (CAS No: 66750-10-5), Mw 295.38

In the present example, the commercially available compounds 1 to 5 (Tokyo Chemical Industry Co., Ltd.) were each mixed in the organic electrolyte 106. Further, for mixing in the organic electrolyte 106, dispersion was performed at the maximum output for about two hours using an ultrasonic cleaner. Furthermore, for the organic electrolyte 106, the one obtained by dissolving LiTFSA in an organic solvent (TEGDME solvent) in a concentration of 1 mol/L was used. Each of the compounds 1 to 5 was dissolved as an additive in a weight of 0.01 wt % in the organic electrolyte 106.

Still further, using La_{0.6}Sr_{0.4}MnO₃ known as the catalyst for air electrode, a lithium air secondary battery cell was manufactured in the following procedure. La_{0.6}Sr_{0.4}MnO₃ was synthesized by a known method using citric acid.

A La_{0.6}Sr_{0.4}MnO₃ powder, a ketjen black powder, and a polyvinylidene fluoride (PVDF) powder were sufficiently mixed at a weight ratio of 10:72:18 in N-methyl-2-pyrrolidone (NMP) using a mixer, thereby manufacturing a slurry. The slurry was coated on a carbon sheet with a diameter of 17 mm. The sheet was placed in a 90° C. vacuum dryer, and was dried overnight, resulting in a gas diffusion type air electrode.

Then, the cell of a cylindrical lithium air secondary battery 100 having the cross sectional structure shown in FIG. 2 was manufactured. The lithium air battery cell was manufactured in a dry air with a dew point of −60° C. or less in the following procedure.

The air electrode 102 prepared by the foregoing method was placed at the inner concave part of the PTFE-coated air electrode support 2, and was fixed with an air electrode fixing PTFE ring 3. Incidentally, the contact part between the air electrode 102 and the air electrode support 2 was not coated with PTFE for establishing an electric contact.

Then, at the surface opposite to the surface at which the air electrode 102 and the atmosphere were in contact with each other, the separator 5 for a lithium air secondary battery was placed at the bottom surface of the concave part. Subsequently, onto the negative electrode fixing washer 7 as shown in FIG. 2, four metallic lithium foils each with a thickness of 150 μm of the negative electrode 104 were stacked concentrically, and crimped. Subsequently, the negative electrode fixing PTFE ring 6 was placed at the opposite concave part opposed to the concave part at which the air electrode 102 was set. At the central part, the negative electrode fixing washer 7 crimped with metallic lithium was further placed. Subsequently, an O ring 9 was placed at the bottom of the air electrode support 2 as shown in FIG. 2.

Thereafter, the inside of the cell (between the air electrode 102 and the negative electrode 104) was filled with the organic electrolyte 106, and was covered with a negative electrode support 11, thereby fixing the whole cell by a cell fixing screw 12. For the organic electrolyte 106, the ferric pyrophosphate-containing organic electrolyte (1 mol/l: LiTFSA/TEGDME solution) was used. Then, finally, the air electrode terminal 4 was set at the air electrode support 2, and the negative electrode terminal 13 was set at the negative electrode support 11.

With a cycle test of a battery, using a charging and discharging measuring system (manufactured by BioLogic), a current was passed at 0.1 mA/cm² with a current density per area of the air electrode 102, and the discharge voltage was measured until the battery voltage was reduced to 2.0 V from the open circuit voltage. The charging test of the battery was performed until the battery voltage reached 4.2 V at the same current density as that during discharging. The charging and discharging test of the battery was performed under normal living environment. The charge and discharge capacities are each expressed as the value (mAh/g) per weight of the air electrode (carbon+oxide+PVDF).

### Test Results of Battery Performances of Lithium Air Secondary Battery of Present Example

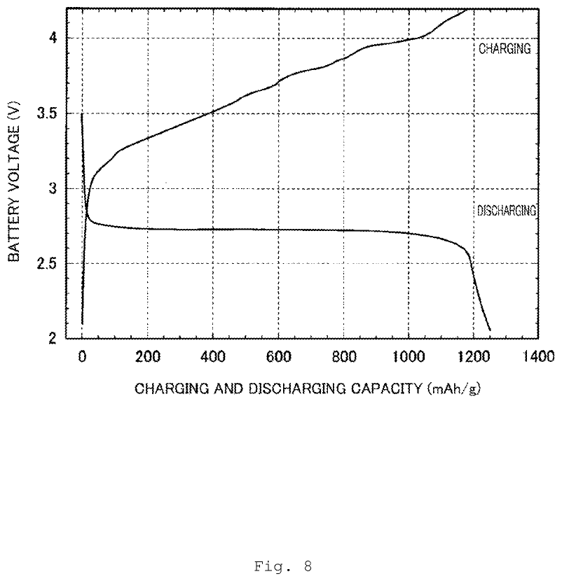

The initial discharging and charging curves of Example 1 are shown in FIG. 8. FIG. 8 indicates that the average discharging voltage is 2.75 V, and the discharge capacity is as large as 1255 mAh/g. Herein, the average charging discharging voltages are defined as the discharging voltage and the charging voltage at the intermediate value of all the discharge capacities in the drawing. Further, the initial charging voltage is 3.73 V, and the charge capacity is 1185 mAh/g almost the same as the discharge capacity, indicating excellent reversibility.

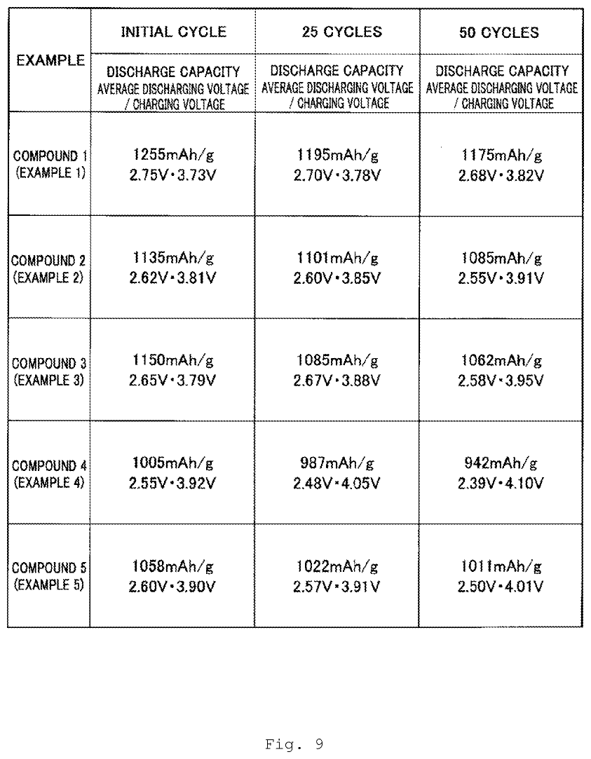

The results of the battery performance tests of Examples 1 to 5 are shown in FIG. 9. In any example, discharging/charging were possible, at the initial time, a discharge capacity as large as more than 1000 mAh/g was shown, and also after 50 cycles, the reduction of the discharge capacity was 10% or less. On the other hand, for each of the discharging voltage/charging voltage, performance reduction of gradual reduction/increase was observed. However, it was confirmed that Example 1 showed the best voltage performances even after 50 cycles. The results of the performance tests indicate that as the effects of addition to the organic electrolyte 106, the activity was higher in the following order.

Compound 1>compound 2 compound 3>compound 5>compound 4

In this order, the correlation with the molecular weight of the compound is observed. This indicates that a compound with a smaller molecular weight exhibits a higher activity. This can be considered due to the following: when a low-molecular-weight compound is added, the increase in viscosity of the electrolyte is suppressed, and hence the diffusion of lithium ions is performed smoothly.

### Test Results of Comparative Examples 1 and 2 with Respect to Present Example

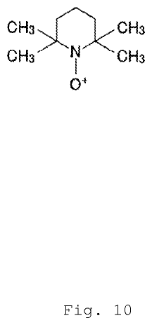

In order to verify the effects of the present example, battery performance tests in the case where no additive is added (Comparative Example 1) and the case using (compound 6, Comparative Example 2) 2,2,6,6-tetramethylpyperidine-1-oxyl (TEMPO, CAS No: 2564-83-2) of a known additive were performed. The structural formula of compound 6 is shown in FIG. 10. Battery manufacturing and evaluation were performed in the same manner as with Examples 1 to 5.

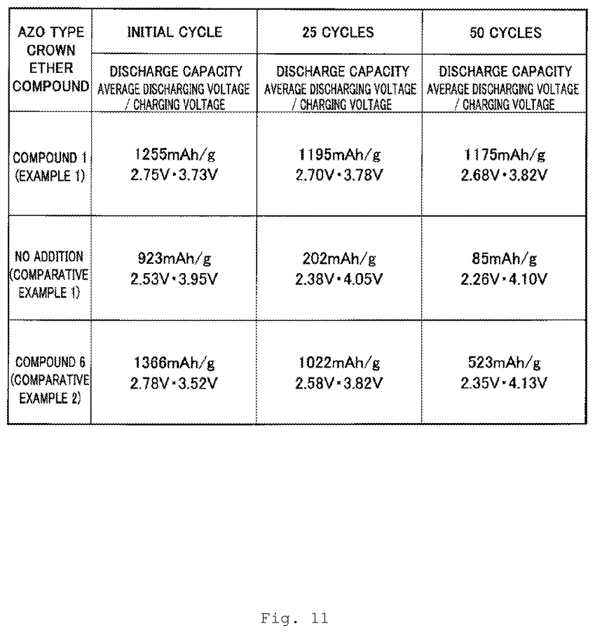

The measurement results of Comparative Examples 1 and 2 are shown in FIG. 11. For comparison, the results of Example 1 are also shown together. In the case of Comparative Example 1 without addition of an additive, the initial capacity was apparently small, and upon repetition of the cycle, a remarkable reduction of the capacity was observed. Further, in the case of TEMPO of Comparative Example 2 of a known additive, it was confirmed that, for the initial cycle, a larger discharge capacity than that of Example 1 and high discharging voltage/low charging voltage were exhibited. However, after 50 cycles, the capacity reduction of about 60% and the reduction of the voltage characteristics were remarkably observed. For this reason, it was confirmed that Example 1 exhibited apparently higher performances than those of Comparative Example 2, and the additives (compounds 1 to 5) of Examples 1 to 5 were verified to have excellent long term stability.

From the description up to this point, in accordance with the present embodiment, the lithium air secondary battery 100 has an air electrode 102 using oxygen in the air as a positive electrode active material, a negative electrode 104 using metallic lithium or a lithium-containing material as a negative electrode active material, and an organic electrolyte 106 placed between the air electrode 102 and the negative electrode 104, and including a lithium salt. The organic electrolyte 106 includes a crown ether compound having an azo group as an additive. For this reason, it is possible to provide a lithium air secondary battery having a large discharge capacity and excellent charging and discharging performances.

Further, in accordance with the present embodiment, the crown ether compound included in the organic electrolyte 106 is any of 1-aza-15-crown-5-ether, 1-aza-18-crown-6-ether, 4,13-diaza-18-crown-6-ether, N,N′-dibenzyl-4,13-diaza-18-crown-6-ether, and N-phenyl aza-15-crown-5-ether. For this reason, it is possible to provide a lithium air secondary battery having a larger discharge capacity and excellent charging and discharging cycling performances.

### INDUSTRIAL APPLICABILITY

By using an azo type crown ether compound as the additive of the organic electrolyte 106, it is possible to manufacture a high-performance lithium air secondary battery 100, which can be effectively used as driving sources for various electronic devices and automobiles.

### REFERENCE SIGNS LIST

100 Lithium air secondary battery
102 Air electrode (positive electrode)
104 Negative electrode
106 Organic electrolyte
2 Air electrode support
3 Air electrode fixing PTFE ring
4 Air electrode terminal
5 Separator
6 Negative electrode fixing PTFE ring
7 Negative electrode fixing washer
9 O ring
11 Negative electrode support
12 Cell fixing screw
13 Negative electrode terminal

1. A lithium air secondary battery, comprising: a positive electrode using oxygen as a positive electrode active material; a negative electrode using metallic lithium or a lithium-containing material as a negative electrode active material; and an organic electrolyte placed between the positive electrode and the negative electrode, and including a lithium salt, wherein the organic electrolyte includes a crown ether compound having an azo group.

2. The lithium air secondary battery according to claim 1, wherein the crown ether compound is any of 1-aza-15-crown-5-ether, 1-aza-18-crown-6-ether, 4,13-diaza-18-crown-6-ether, N,N′-dibenzyl-4,13-diaza-18-crown-6-ether, and N-phenyl aza-15-crown-5-ether.
